(19) United States
(12) Patent Application Publication
(10) Pub. No.: US 2019/0275052 A1
(43) Pub. Date: Sep. 12, 2019

(54) COMBINATION THERAPY OF AN HBV CAPSID ASSEMBLY INHIBITOR AND A NUCLEOS(T)IDE ANALOGUE

(71) Applicant: Hoffmann-La Roche Inc., Little Falls, NJ (US)

(72) Inventors: Isabel Najera, Basel (CH); Steffen Wildum, Basel (CH)

(73) Assignee: Hoffmann-La Roche Inc., Little Falls, NJ (US)

(21) Appl. No.: 16/282,481

(22) Filed: Feb. 22, 2019

Related U.S. Application Data
(63) Continuation of application No. PCT/EP2017/070984, filed on Aug. 21, 2017.

(30) Foreign Application Priority Data
Aug. 24, 2016 (EP) 16185439.3

(51) Int. Cl. A61K 31/5377; A61K 31/522; A61P 31/20; A61K 31/506; A61K 31/675; A61K 31/7072; A61K 31/683

(52) U.S. Cl. CPC A61K 31/5377; A61K 31/522; A61P 31/20; A61K 31/683; A61K 31/675; A61K 31/7072; A61K 31/506

(57) ABSTRACT

The present invention is directed to compositions and methods for treating hepatitis B virus infection. In particular, the present invention is directed to a combination therapy comprising administration of an HBV capsid assembly inhibitor and a nucleoside or nucleotide analogue for use in the treatment or prophylaxis of chronic hepatitis B patient.

### CROSS REFERENCE TO RELATED APPLICATIONS

This application is a continuation of International Application No. PCT/EP2017/070984, filed Aug. 21, 2017, which claims priority to European Application No. 16185439.3 filed Aug. 24, 2016, each of which are incorporated herein by reference in its entirety.

### SEQUENCE LISTING

This application contains a Sequence Listing which has been submitted via EFS-Web and is hereby incorporated by reference in its entirety. Said ASCII copy, created on Feb. 15, 2019, is named Sequence Listing and is 791 bytes in size.

The present invention is directed to compositions and methods for treating hepatitis B virus infection. In particular, the present invention is directed to a combination therapy comprising administration of an HBV capsid assembly inhibitor and a nucleos(t)ide analogue for use in the treatment or prophylaxis of hepatitis B virus infections.

### FIELD OF THE INVENTION

Chronic infection of Hepatitis B virus (HBV) is a serious public health problem worldwide, with more than 240 million people chronically infected worldwide. HBV belongs to the Hepadnaviridae family of viruses. Following entry into hepatocyte, its viral genome is delivered into nucleus where a covalently closed circular DNA (cccDNA) is formed through DNA repair of partially double-stranded viral genome. cccDNA serves as the template for transcription of viral RNAs. Viral pre-genomic RNA interacts with other two viral components, capsid protein and polymerase to form capsid particles where viral DNA replication occurs. HBV has an icosahedral core comprising of 240 copies of the capsid (or core) protein. The predominant biological function of capsid protein is to act as a structural protein to encapsidate pre-genomic RNA and form immature capsid particles in the cytoplasm. This step is prerequisite for viral DNA replication. When a near full-length relaxed circular DNA is formed through reverse-transcription of viral pregenomic RNA, an immature capsid becomes a mature capsid. Most copies of the encapsidated genome efficiently associate with cellular lipids and viral envelope proteins (S, M, and L) for virion assembly and secretion. However, non-infectious particles are also produced that greatly outnumber the infectious virions. These empty, enveloped particles are referred to as subviral particles (SVPs). The S, M, and L envelope proteins are expressed from a single ORF (open reading frame) that contains three different start codons. All three proteins share a 226aa sequence, the S-domain, at their C-termini. S-domain contains the HBsAg epitope (Lambert, C. & R. Prange. Virol J, 2007, 4, 45).

Many observations showed that several HBV viral proteins could counteract the initial host cellular response by interfering with the viral recognition signaling system. Among these, the excessive secretion of HBV empty subviral particles may participate to the maintenance of the immunological tolerant state observed in chronically infected patients (CHB). The persistent exposure to HBsAg and other viral antigens can lead to HBV-specific T-cell deletion or to progressive functional impairment (Kondo et al. Journal of Immunology 1993, 150, 4659-4671; Kondo et al. Journal of Medical Virology 2004, 74, 425-433; Fisicaro et al. Gastroenterology, 2010, 138, 682-93). Moreover HBsAg has been reported to suppress the function of immune cells such as monocytes, dendritic cells (DCs) and natural killer (NK) cells by direct interaction (Op den Brouw et al. Immunology, 2009b, 126, 280-9; Woltman et al. PLoS One, 2011, 6, e15324; Shi et al. J Viral Hepat. 2012, 19, e26-33; Kondo et al. ISRN Gasteroenterology, 2013, Article ID 935295).

It has been well studied that HBV capsid protein plays essential roles in HBV replication. Heteroaryldihydropyrimidines or HAP, including compounds named Bay 41-4109, Bay 38-7690 and Bay 39-5493, were discovered in a tissue culture-based screening (Deres K. et al. Science 2003, 893). These HAP analogs act as synthetic allosteric activators and are able to induce aberrant capsid formation that leads to degradation of the core protein. HAP analogs also reorganized core protein from preassembled capsids into noncapsid polymers, presumably by interaction of HAP with dimers freed during capsid ‘breathing’, the transitory breaking of individual inter-subunit bonds. Bay 41-4109 was administered to HBV infected transgenic mouse model or humanized mouse models and demonstrated in vivo efficacy with HBV DNA reduction (Deres K. et al. Science 2003, 893; Brezillon N. et al. PLoS ONE 2011, e25096). Furthermore, more HAP analogs with potential different mechanism were disclosed in Roche patent WO2013/144129, WO2014/037480, WO 2014/184328 and WO2015/132276.

Current HBV therapies include nucleos(t)ide analogues (e.g. Lamivudine, Adefovir, Tenofovir, Telbivudine and Entecavir), which target viral polymerase by inhibiting HBV polymerase reverse transcription activities. This leads to a decreased viral load and an abolishment of HBV progeny production, but cccDNAs remain intact, and syntheses of all viral proteins and RNAs, and HBsAg level are not affected in the infected hepatocytes. Even with prolonged therapy, nucleos(t)ide analogues have demonstrated very low rates of HBsAg clearance comparable to those observed naturally (Janssen et al. Lancet, 2005, 365, 123-9; Marcellin et al. N. Engl. J. Med., 2004, 351, 1206-17; Buster et al. Hepatology, 2007, 46, 388-94).

HBsAg is a biomarker for prognosis and treatment response in CHB. Now, the standard of clinic cure of HBV infection is the loss and/or seroconversion of HBsAg. Even though nucleos(t)ide analogues are available to HBV patients, the majority (around or more than 90%) of treated patients fail to achieve this goal. The Hepatitis B virus (HBV) infection remains a major health problem worldwide which concerns an estimated 240 million chronic carriers who have a higher risk of liver cirrhosis and hepatocellular carcinoma. Hence, there is certainly an unmet medical need for treatments with improved success rate of inducing HBsAg loss, and/or HBeAg loss, and/or HBV-DNA reduction, and/or HBV clearance, and/or seroconversion, and/or normalization of ALT, and/or promoting the production of anti-HBs to address the Hepatitis B virus (HBV) infections.

### SUMMARY OF THE INVENTION

The present invention relates to a pharmaceutical composition comprising an HBV capsid assembly inhibitor and a nucleoside or nucleotide analogue, in a pharmaceutically acceptable carrier for the treatment or prophylaxis of HBV infection.

The “HBV capsid assembly inhibitor” herein is a compound of formula (I) or (II), or any one of the compounds disclosed in patent WO2013/144129, WO2014/037480, WO 2014/184328 and WO2015/132276; particularly the “HBV capsid assembly inhibitor” herein is (S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid; 3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid; 3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid; 4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8,8a-tetrahydro-1H-imidazo[1,5-a]pyrazin-2-yl]-3,3-dimethyl-butanoic acid; or 3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid; or pharmaceutically acceptable salt, or enantiomer, or diastereomer thereof.

In one embodiment, the “nucleoside or nucleotide analogue” is any nucleoside or nucleotide analogue known to those skilled in the art. In a further embodiment, the “nucleoside or nucleotide analogue” is Entecavir, Lamivudine, Adefovir dipivoxil, Telbivudine, Clevudine, Tenofovir disoproxil or Tenofovir disoproxil fumarate. Particularly the “nucleoside or nucleotide analogue” is Entecavir.

### BRIEF DESCRIPTION OF THE FIGURE(S)

FIG. 1: Isobologram of FIC for the pair-wise checkerboard combination of Compound 1 and Compound 6 (at the 50% effect level). The diagonal lane connecting points (0, 1) and (1, 0) represents additivity (CI=1). Data points below this lane show synergism, data points above show antagonism. Shown are mean values from 3 independent experiments.

FIG. 2: Isobologram of FIC for the pair-wise checkerboard combination of Compound 2 and Compound 6 (at the 50% effect level). The diagonal lane connecting points (0, 1) and (1, 0) represents additivity (CI=1). Data points below this lane show synergism, data points above show antagonism. Shown are mean values from 3 independent experiments.

FIG. 3: Isobologram of FIC for the pair-wise checkerboard combination of Compound 3 and Compound 6 (at the 50% effect level). The diagonal lane connecting points (0, 1) and (1, 0) represents additivity (CI=1). Data points below this lane show synergism, data points above show antagonism. Shown are mean values from 3 independent experiments.

FIG. 4: Isobologram of FIC for the pair-wise checkerboard combination of Compound 4 and Compound 6 (at the 50% effect level). The diagonal lane connecting points (0, 1) and (1, 0) represents additivity (CI=1). Data points below this lane show synergism, data points above show antagonism. Shown are mean values from 3 independent experiments.

FIG. 5: Isobologram of FIC for the pair-wise checkerboard combination of Compound 5 and Compound 6 (at the 50% effect level). The diagonal lane connecting points (0, 1) and (1, 0) represents additivity (CI=1). Data points below this lane show synergism, data points above show antagonism. Shown are mean values from 3 independent experiments.

### DETAILED DESCRIPTION OF THE INVENTION

### Definitions

Unless defined otherwise, all technical and scientific terms used herein have the same meaning as commonly understood by one of ordinary skill in the art to which this invention pertains.

As used herein, the term “C₁₋₆alkyl” refers to a monovalent linear or branched saturated hydrocarbon group of 1 to 6 carbon atoms. In particular embodiments, C₁₋₆alkyl has 1 to 6 carbon atoms, and in more particular embodiments 1 to 4 carbon atoms. Examples of C₁₋₆alkyl include methyl, ethyl, propyl, isopropyl, n-butyl, iso-butyl, sec-butyl or tert-butyl.

As used herein, the term “halo” or “halogen” are used interchangeably herein and refer to fluoro, chloro, bromo, or iodo. Halogen is particularly fluorine, chlorine or bromine.

As used herein, the term “C₁₋₆alkoxy” refers to a group of C₁₋₆alkyl-O—, wherein the “C₁₋₆alkyl” is as defined above; for example methoxy, ethoxy, propoxy, iso-propoxy, n-butoxy, iso-butoxy, 2-butoxy, tert-butoxy and the like. Particular “C₁₋₆alkoxy” groups are methoxy and ethoxy and more particularly methoxy.

As used herein, the term “C₃₋₇cycloalkyl” refers to a saturated carbon ring containing from 3 to 7 carbon atoms, particularly from 3 to 6 carbon atoms, for example, cyclopropyl, cyclobutyl, cyclopentyl, cyclohexyl, cycloheptyl and the like. Particular “C₃₋₇cycloalkyl” groups are cyclopropyl, cyclopentyl and cyclohexyl.

As used herein, the term “CₓH₂ₓ” alone or in combination signifies a saturated, linear or branched chain alkyl group containing 1 to 6, particularly 1 to 4 carbon atoms.

As used herein, the term “diastereomer” refers to a stereoisomer with two or more centers of chirality and whose molecules are not mirror images of one another. Diastereomers have different physical properties, e.g. melting points, boiling points, spectral properties, activities and reactivities.

As used herein, the term “enantiomers” refers to two stereoisomers of a compound which are non-superimposable mirror images of one another.

As used herein, the term “pharmaceutically acceptable salts” refers to salts which are not biologically or otherwise undesirable. Pharmaceutically acceptable salts include both acid and base addition salts. As used herein, the term “prodrug” refers to a form or derivative of a compound which is metabolized in vivo, e.g., by biological fluids or enzymes by a subject after administration, into a pharmacologically active form of the compound in order to produce the desired pharmacological effect. Prodrugs are described e.g. in the Organic Chemistry of Drug Design and Drug Action by Richard B. Silverman, Academic Press, San Diego, 2004, Chapter 8 Prodrugs and Drug Delivery Systems, pp. 497-558.

The term “pharmaceutically acceptable acid addition salt” refers to those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” refers to those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

As used herein, “hepatitis B virus” or “HBV” refers to a member of the Hepadnaviridae family having a small double-stranded DNA genome of approximately 3,200 base pairs and a tropism for liver cells. “HBV” includes hepatitis B virus that infects any of a variety of mammalian (e.g., human, non-human primate, etc.) and avian (duck, etc.) hosts. “HBV” includes any known HBV genotype, e.g., serotype A, B, C, D, E, F, and G; any HBV serotype or HBV subtype; any HBV isolate; HBV variants, e.g., HBeAg-negative variants, drug-resistant HBV variants (e.g., lamivudine-resistant variants; adefovir-resistant mutants; tenofovir-resistant mutants; entecavir-resistant mutants; etc.); and the like.

As used herein, “HBV DNA” refers to DNA material of HBV.

As used herein, “HBsAg” refers to hepatitis B surface antigen.

As used herein, “HBeAg” refers to hepatitis B e antigen.

As used herein, the “nucleoside analogue” refers to nucleosides which contain a nucleobase analogue and a sugar and nucleotides which contain a nucleobase analogue, a sugar and one to three phosphate groups. Nucleoside analogue drugs include but are not limited to deoxyadenosine analogues (Didanosine and Vidarabine), adenosine analogues (BCX4430), deoxycytidine analogues (Cytarabine, Emtricitabine, Lamivudine and Zalcitabine), guanosine and deoxyguanosine analogues (Abacavir, Aciclovir and Entecavir), thymidine and deoxythymidine analogues (Stavudine, Telbivudine and Zidovudine) and deoxyuridine analogues (Idoxuridine and Trifluridine). “Nucleotide analogue” drugs include Adefovir dipivoxil (ADV) and Tenofovir disoproxil fumarate (TDF).

The term “Nucleobase” means any nitrogen-containing heterocyclic moiety capable of forming Watson-Crick-type hydrogen bonds and stacking interactions in pairing with a complementary nucleobase or nucleobase analogue (i.e., derivatives of nucleobases) when that nucleobase is incorporated into a polymeric structure. “Heterocyclic” refers to a molecule with a ring system in which one or more ring atom is a heteroatom, e.g., nitrogen, oxygen, or sulfur (i.e., not carbon).

The term “therapeutically effective amount” refers to an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgment of the attending medical or veterinary practitioner, and other factors. The present invention relates to a pharmaceutical composition comprising an HBV capsid assembly inhibitor and a nucleos(t)ide analogue, in a pharmaceutically acceptable carrier.

Compounds of the general formula (I) and (II) which contain one or several chiral centers can either be present as racemates, diastereomeric mixtures, or optically active single isomers. Particularly, diastereomeric salts which can be separated by crystallization are formed from the racemic mixtures by reaction with an optically active acid such as e.g. D- or L-tartaric acid, mandelic acid, malic acid, lactic acid or camphorsulfonic acid.

The present invention relates to a pharmaceutical composition comprising an HBV capsid assembly inhibitor and a nucleoside or nucleotide analogue, in a pharmaceutically acceptable carrier.

In one embodiment of present invention, the HBV capsid assembly inhibitor is a compound of formula (I):

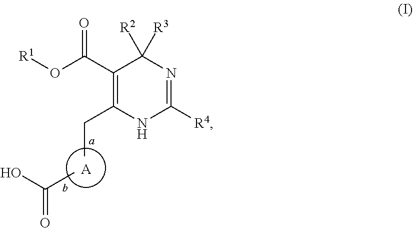

wherein

R¹ is C₁₋₆alkyl or trifluoromethyl-CₓH₂ₓ—, wherein x is 1, 2, 3, 4, 5 or 6;

One of R² and R³ is phenyl, which is once or twice or three times substituted by C₁₋₆alkyl, cyano or halogen; and the other one is hydrogen or deuterium;

R⁴ is phenyl, thiazolyl, oxazolyl, imidazolyl, thienyl or pyridinyl, which is unsubstituted or substituted by C₁₋₆alkyl, C₁₋₆alkylsulfanyl, halogen or cycloalkyl, wherein C₁₋₆alkyl can be further optionally substituted with halogen;
A is

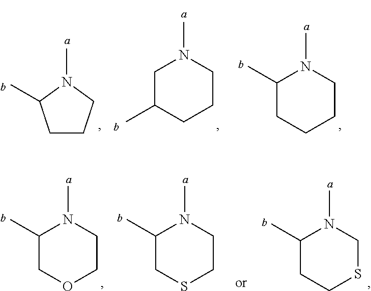

which is unsubstituted or substituted by groups selected from C₁₋₆alkyl, deuterium and halogen;

or pharmaceutically acceptable salt, or enantiomer, or diastereomer thereof.

More particularly the HBV capsid assembly inhibitor according to present invention relates to (S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid; or any other compound disclosed in patent WO2014/037480; or pharmaceutically acceptable salt, or enantiomer, or diastereomer thereof. Compounds of formula (I) and compound 1 can be obtained by the synthetic procedures described in WO2014/037480.

In another embodiment of present invention, the HBV capsid assembly inhibitor is a compound of formula (II):

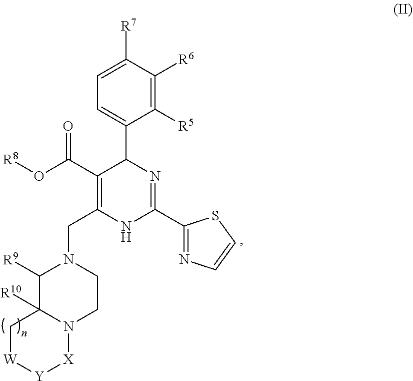

wherein

R⁵ is hydrogen, halogen or C₁₋₆alkyl;
R⁶ is hydrogen or halogen;
R⁷ is hydrogen or halogen;
R⁸ is C₁₋₆alkyl;
R⁹ is hydrogen, hydroxyC₁₋₆alkyl, aminocarbonyl, C₁₋₆alkoxycarbonyl or carboxy;
R¹⁰ is hydrogen, C₁₋₆alkoxycarbonyl or carboxy-CₘH₂ₘ—;
X is carbonyl or sulfonyl;
Y is —CH₂—, —O— or —N(R¹¹)—,
wherein R¹¹ is hydrogen, C₁₋₆alkyl, haloC₁₋₆alkyl, C₃₋₇cycloalkyl-CₘH₂ₘ—, C₁₋₆alkoxycarbonyl-CₘH₂ₘ—, —CₜH₂ₜ—COOH, -haloC₁₋₆alkyl-COOH, —(C₁₋₆alkoxy)C₁₋₆alkyl-COOH, —C₁₋₆alkyl-O—C₁₋₆alkyl-COOH, —C₃₋₇cycloalkyl-CₘH₂ₘ—COOH, —CₘH₂ₘ—C₃₋₇cycloalkyl-COOH, hydroxy-CₜH₂ₜ—, carboxyspiro[3.3]heptyl or carboxyphenyl-CₘH₂ₘ—, carboxypyridinyl-CₘH₂ₘ—;
   W is —CH₂—, —C(C₁₋₆alkyl)₂-, —O— or carbonyl;
   n is 0 or 1;
   m is 0-7;
   t is 1-7;

or pharmaceutically acceptable salt, or enantiomer or diastereomer thereof.

More particularly the HBV capsid assembly inhibitor according to present invention relates to 3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid; 3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid; 4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid; or 3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid; or any other compound disclosed in patent WO2015/132276; or a pharmaceutically acceptable salt, enantiomer or diastereomer thereof. Compounds of formula (II) and compound 2, 3, 4 and 5 can be obtained by the synthetic procedures described in WO2015/132276.

In another embodiment of present invention, the HBV capsid assembly inhibitor used in the combination with nucleoside or nucleotide is any compound selected from patent WO2008154817, WO2008154819, WO2014029193, WO2015074546, CN103664897 and CN103664925.

In one embodiment of the present invention, the suitable nucleoside or nucleotide analogue is Entecavir, Lamivudine, Adefovir dipivoxil, Telbivudine, Clevudine, Tenofovir disoproxil or Tenofovir disoproxil fumarate. Particularly the nucleoside analogue is Entecavir.

In one embodiment of the present invention, the pharmaceutical composition comprises an HBV capsid assembly inhibitor and a nucleoside or nucleotide analogue, wherein the HBV capsid assembly inhibitor and the nucleoside or nucleotide analogue are independently selected from Table 1.

TABLE 1
List of HBV capsid assembly inhibitors and nucleoside or nucleotide analogues

| Compound | | | |
|---|---|---|---|
| Number | Class | Compound Name/Product Name | Structure |
| 1 | HBV capsid assembly inhibitor | (S)-4-[(R)-6-(2-Chloro-4-fluoro- phenyl)-5-methoxycarbonyl-2- thiazol-2-yl-3,6-dihydro- pyrimidin-4-ylmethyl]- morpholine-3-carboxylic acid | 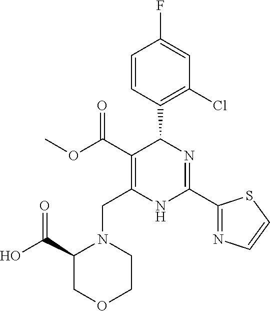 [SMILES: COC(=O)C1=C(CN2CCOC[C@H]2C(=O)O)NC(c2nccs2)=N[C@H]1c1ccc(F)cc1Cl] |
| 2 | HBV capsid assembly inhibitor | 3-[(8aS)-7-[[(4S)5- ethoxycarbonyl-4-(3-fluoro-2- methyl-phenyl)-2-thiazol-2-yl- 1,4-dihydropyrimidin-6- yl]methyl]-3-oxo-5,6,8,8a- tetrahydro-1H-imidazo[1,5- a]pyrazin-2-yl)-2,2-dimethyl- propanoic acid | 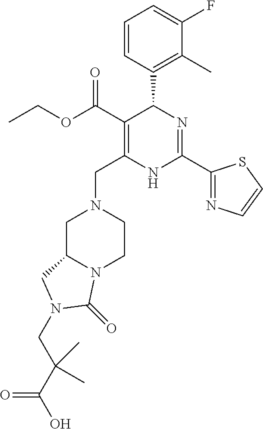 [SMILES: CCOC(=O)C1=C(CN2CCN3C(=O)N(CC(C)(C)C(=O)O)C[C@@H]3C2)NC(c2nccs2)=N[C@H]1c1cccc(F)c1C] |
| 3 | HBV capsid assembly inhibitor | 3-[(8aS)-7-[[(4R)-4-(2-chloro-3- fluoro-phenyl)-5-ethoxycarbonyl- 2-thiazol-2-yl-1,4- dihydropyrimidin-6-yl]methyl]-3- oxo-5,6,8,8a-tetrahydro-1H- imidazo[1,5-a]pyrazin-2-yl]-2,2- dimethyl-propanoic acid | 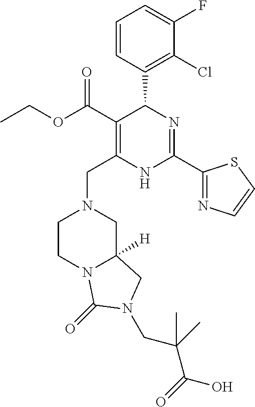 [SMILES: CCOC(=O)C1=C(CN2CCN3C(=O)N(CC(C)(C)C(=O)O)C[C@@H]3C2)NC(c2nccs2)=N[C@H]1c1cccc(F)c1Cl] |
| 4 | HBV capsid assembly inhibitor | 4-[(8aS)-7-[[(4R)-4-(2-chloro-4- fluoro-phenyl)-5- methoxycarbonyl-2-thiazol-2-yl- 1,4-dihydropyrimidin-6- yl]methyl]-3-oxo-5,6,8,8a- tetrahydro-1H-imidazo[1,5- a]pyrazin-2-yl]-3,3-dimethyl- butanoic acid | 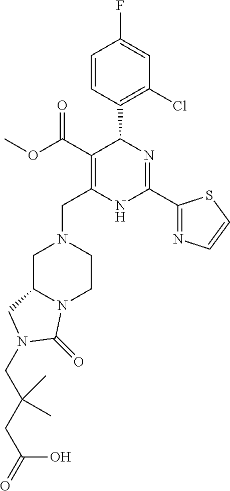 [SMILES: COC(=O)C1=C(CN2CCN3C(=O)N(CC(C)(C)CC(=O)O)C[C@@H]3C2)NC(c2nccs2)=N[C@H]1c1ccc(F)cc1Cl] |
| 5 | HBV capsid assembly inhibitor | 3-[(8aS)-7-[[(4R)-4-(2-chloro-4- fluoro-phenyl)-5- methoxycarbonyl-2-thiazol-2-yl- 1,4-dihydropyrimidin-6- yl]methyl]-3-oxo-5,6,8,8a- tetrahydro-1H-imidazo[1,5- a]pyrazin-2-yl]-2,2-dimethyl- propanoic acid | 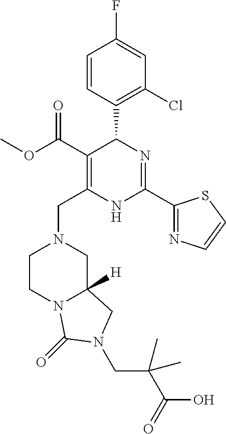 [SMILES: COC(=O)C1=C(CN2CCN3C(=O)N(CC(C)(C)C(=O)O)C[C@H]3C2)NC(c2nccs2)=N[C@H]1c1ccc(F)cc1Cl] |
| 6 | Nucleoside | Entecavir | 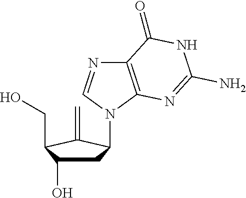 |

Nucleoside/nucleotide analogue herein is also selected from Lamivudine, Adefovir dipivoxil, Telbivudine, Clevudine, Tenofovir disoproxil and Tenofovir disoproxil fumarate.

More particularly, the present invention relates to a pharmaceutical composition comprising HBV capsid assembly inhibitor and a nucleoside or nucleotide analogue which is selected from any one of the following combinations:

Compound 1 and Entecavir; Compound 2 and Entecavir;

Compound 3 and Entecavir; Compound 4 and Entecavir;

Compound 5 and Entecavir; Compound 1 and Lamivudine;

Compound 2 and Lamivudine; Compound 3 and Lamivudine;

Compound 4 and Lamivudine; Compound 5 and Lamivudine;

Compound 1 and Adefovir dipivoxil; Compound 2 and Adefovir dipivoxil;

Compound 3 and Adefovir dipivoxil; Compound 4 and Adefovir dipivoxil;

Compound 5 and Adefovir dipivoxil; Compound 1 and Telbivudine;

Compound 2 and Telbivudine; Compound 3 and Telbivudine;

Compound 4 and Telbivudine; Compound 5 and Telbivudine;

Compound 1 and Clevudine; Compound 2 and Clevudine;

Compound 3 and Clevudine; Compound 4 and Clevudine;

Compound 5 and Clevudine; Compound 1 and Tenofovir disoproxil;

Compound 2 and Tenofovir disoproxil; Compound 3 and Tenofovir disoproxil;

Compound 4 and Tenofovir disoproxil; Compound 5 and Tenofovir disoproxil;

Compound 1 and Tenofovir disoproxil fumarate;

Compound 2 and Tenofovir disoproxil fumarate;

Compound 3 and Tenofovir disoproxil fumarate;

Compound 4 and Tenofovir disoproxil fumarate; and

Compound 5 and Tenofovir disoproxil fumarate;

in a pharmaceutically acceptable carrier.

Any one of Compounds 1 to 5 of the aforementioned combinations can be replaced by its corresponding pharmaceutically acceptable salt, enantiomer or diastereomer, which is another aspect of this invention.

More particularly, the present invention relates to a pharmaceutical composition consists of:
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Entecavir;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Entecavir;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Entecavir;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8,8a-tetrahydro-1H-imidazo[1,5-a]pyrazin-2-yl]-3,3-dimethyl-butanoic acid and Entecavir;
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Entecavir;
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Lamivudine;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Lamivudine;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Lamivudine;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Lamivudine;
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Lamivudine;
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Adefovir dipivoxil;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Adefovir dipivoxil;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid and Adefovir dipivoxil;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Adefovir dipivoxil;
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Adefovir dipivoxil;
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Telbivudine;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Telbivudine;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Telbivudine;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Telbivudine;
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid and Telbivudine;
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Clevudine;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Clevudine;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid and Clevudine;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Clevudine;
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Clevudine;
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Tenofovir disoproxil;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Tenofovir disoproxil;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Tenofovir disoproxil;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Tenofovir disoproxil;
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid and Tenofovir disoproxil;
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Tenofovir disoproxil fumarate;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid and Tenofovir disoproxil fumarate;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Tenofovir disoproxil fumarate;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8,8a-tetrahydro-1H-imidazo[1,5-a]pyrazin-2-yl]-3,3-dimethyl-butanoic acid and Tenofovir disoproxil fumarate; or
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Tenofovir disoproxil fumarate;
   in a pharmaceutically acceptable carrier.

In one embodiment of the present invention, the pharmaceutical composition consists of:
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Entecavir;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid and Entecavir;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Entecavir;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Entecavir; or
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Entecavir;
   in a pharmaceutically acceptable carrier.

Typical dosages of an HBV capsid assembly inhibitor and/or a nucleos(t)ide analogue can be in various ranges, and where indicated by in vitro responses in an animal model, can be reduced by up to about one order of magnitude concentration or amount. Thus, the actual dosage will depend upon the judgment of the physician, the condition of the patient, and the effectiveness of the therapeutic method based on the in vitro responsiveness of the appropriate animal models.

Another embodiment of the present invention relates to a method for manufacturing a medicament for treatment or prophylaxis of hepatitis B virus infection, characterized in that an HBV capsid assembly inhibitor and a nucleoside or nucleotide analogue are used in the medicament.

A further embodiment of the present invention relates to the method for manufacturing a medicament for treatment or prophylaxis of hepatitis B virus infection, characterized in that an HBV capsid assembly inhibitor and a nucleoside or nucleotide analogue are co-administered in the same formulation or different formulation.

For purposes of the present invention, “co-administer” refers to any administration of an HBV capsid assembly inhibitor and a nucleoside or nucleotide analogue as the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. Also, the two active agents can be administered either at the same time, or sequentially.

The pharmaceutical composition of an HBV capsid assembly inhibitor and a nucleoside or nucleotide analogue can be administered with various pharmaceutically acceptable inert carriers in the form of tablets, capsules, lozengens, troches, hard candies, powders, sprays, creams, salves, suppositories, jellies, gels, pastes, lotions, ointments, elixirs, syrups, and the like. Administration of such dosage forms can be carried out in single or multiple doses. Carries include solid diluents of fillers, sterile aqueous media and various non-toxic organic solvents. Administration of such dosage forms can be carried out through, but not limited to, oral administration, parenteral administration, veterinary administration.

A further embodiment of the present invention relates to the method for manufacturing a medicament for treatment or prophylaxis of hepatitis B virus infection, characterized in that an HBV capsid assembly inhibitor and a nucleoside or nucleotide analogue are intended for administration to a subject by the same route or different routes.

A further embodiment of the present invention relates to the method for manufacturing a medicament for treatment or prophylaxis of hepatitis B virus infection, characterized in that an HBV capsid assembly inhibitor and a nucleoside or nucleotide analogue are intended for administration to a subject by parenteral or oral administration.

A further embodiment of the present invention relates to the method for manufacturing a medicament for treatment or prophylaxis of hepatitis B virus infection, characterized in that the administration of an HBV capsid assembly inhibitor and a nucleoside or nucleotide analogue to a subject is simultaneous or sequential. In any of the methods of the present invention, the administration of agents simultaneously can be performed by separately or sequentially administering agents at the same time, or together as a fixed combination. Also, in any of the methods of the present invention, the administration of agents separately or sequentially can be in any order.

Another embodiment of the present invention relates to the method for manufacturing a medicament for treatment or prophylaxis of hepatitis B virus infection, characterized in that the HBV capsid assembly inhibitor used in the medicament is a compound of formula (I) or formula (II), or pharmaceutically acceptable salt, enantiomer or diastereomer thereof. Particularly, the HBV capsid assembly inhibitor is
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8,8a-tetrahydro-1H-imidazo[1,5-a]pyrazin-2-yl]-3,3-dimethyl-butanoic acid; or
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid;

or a pharmaceutically acceptable salt, enantiomer or diastereomer thereof.

Another embodiment of the present invention relates to the method for manufacturing a medicament for treatment or prophylaxis of hepatitis B virus infection, characterized in that the nucleoside or nucleotide analogue used in the medicament is Entecavir, Lamivudine, Adefovir dipivoxil, Telbivudine, Clevudine, Tenofovir disoproxil or Tenofovir disoproxil fumarate. Particularly the nucleoside analogue is Entecavir.

Another embodiment of present invention relates to the method for manufacturing a medicament for treatment or prophylaxis of hepatitis B virus infection, characterized in that the HBV capsid assembly inhibitor and the nucleoside or nucleotide analogue used in the medicament are
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Entecavir;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Entecavir;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Entecavir;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Entecavir; or
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid and Entecavir.

Another embodiment of the present invention relates to a kit comprising a container comprising an HBV capsid assembly inhibitor and a nucleoside or nucleotide analogue, said kit can further comprise a sterile diluent.

A further embodiment of the present invention relates to the said kit, wherein the kit can further comprise a package insert comprising printed instructions directing the use of a combined treatment of an HBV capsid assembly inhibitor and a nucleoside or nucleotide analogue as a method for treatment or prophylaxis of hepatitis B virus infection.

Another embodiment of present invention relates to the said kit, wherein the HBV capsid assembly inhibitor used in the container is
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8,8a-tetrahydro-1H-imidazo[1,5-a]pyrazin-2-yl]-3,3-dimethyl-butanoic acid; or
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid;

or a pharmaceutically acceptable salt, enantiomer or diastereomer thereof.

Another embodiment of present invention relates to the said kit, wherein the nucleoside or nucleotide analogue used in the said kit is Entecavir, Lamivudine, Adefovir dipivoxil, Telbivudine, Clevudine, Tenofovir disoproxil or Tenofovir disoproxil fumarate. Particularly the nucleoside analogue is Entecavir.

Another embodiment of present invention relates to the said kit, characterized in that the HBV capsid assembly inhibitor and the nucleoside or nucleotide analogue used in the container are
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Entecavir;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Entecavir;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Entecavir;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Entecavir; or
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid and Entecavir.

Another embodiment of present invention relates to a method for the treatment or prophylaxis of hepatitis B virus infection, comprising administration to a subject with an effective first amount of an HBV capsid assembly inhibitor, or pharmaceutically acceptable salt, enantiomer or diastereomer thereof; and a second amount of a nucleoside or nucleotide analogue; or vice versa; wherein the HBV capsid assembly inhibitor is
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8,8a-tetrahydro-1H-imidazo[1,5-a]pyrazin-2-yl]-2,2-dimethyl-propanoic acid;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid; or
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid;

or a pharmaceutically acceptable salt, enantiomer or diastereomer thereof.

Another embodiment of present invention relates to a method for the treatment or prophylaxis of hepatitis B virus infection, comprising administration to a subject with an effective first amount of an HBV capsid assembly inhibitor, or pharmaceutically acceptable salt, enantiomer or diastereomer thereof; and a second amount of a nucleoside or nucleotide analogue; or vice versa; wherein the nucleoside or nucleotide analogue is Entecavir, Lamivudine, Adefovir dipivoxil, Telbivudine, Clevudine, Tenofovir disoproxil or Tenofovir disoproxil fumarate. Particularly the nucleoside analogue is Entecavir.

Another embodiment of present invention relates to a method for the treatment or prophylaxis of hepatitis B virus infection, characterized in that the HBV capsid assembly inhibitor and the nucleoside or nucleotide analogue used in the method are
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Entecavir;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Entecavir;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Entecavir;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8,8a-tetrahydro-1H-imidazo[1,5-a]pyrazin-2-yl]-3,3-dimethyl-butanoic acid and Entecavir; or
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Entecavir.

Another embodiment of present invention relates to use of pharmaceutical composition herein mentioned above as an antiviral medicament, in particular as the medicament for treatment or prophylaxis of hepatitis B virus infection.

Another embodiment of present invention relates to the use of an HBV capsid assembly inhibitor and a nucleoside or nucleotide analogue for the manufacture of pharmaceutical composition herein mentioned above as an antiviral medicament, in particular the medicament for treatment or prophylaxis of hepatitis B virus infection.

### EXAMPLES

The invention will be more fully understood by reference to the following examples. They should not, however, be construed as limiting the scope of the invention.

### Abbreviations

CI Combination index

CTG CellTiter-Glo®

DMSO Dimethyl sulfoxide

FBS Fetal Bovine Serum

FIC Fractional inhibition concentration

GE Genome equivalent

HBsAg Hepatitis B surface antigen

μM Micromolar

Min Minute

Nucleos(t)ide nucleoside or nucleotide

PBS Phosphate buffered saline

PEG Polyethyleneglycol

Pen/Strep Penicillin/Streptomycin

qPCR Real-time quantitative polymerase chain reaction

SD Standard deviation

Sec Second

UDG Uracil DNA glycosylase Example 1

### Combination of an HBV Capsid Assembly Inhibitor and a Nucleoside or Nucleotide Analogue in HBV Infected HepaRG Cells

### Material and Methods

### Virus and Cells

HepG2.2.15 cells were cultured in DMEM+Glutamax I (Gibco, #21885) supplemented with 10% FBS, 1% Pen/Strep (Gibco, #15140) and G-418 (250 μg/mL) and used for production of infectious HBV (genotype D). 90% confluent cells from three T175 flasks were trypsinized and transferred into one collagen coated hyperflask (550 mL). Once the cells are confluent, medium was changed to DMEM+Glutamax I with 1% DMSO and 2.5% FBS. Once the cells were slightly over confluent, medium was changed to DMEM/F12+Glutamax I (Gibco, #31331) supplemented with MEM non-essential amino acids (6 mL, Gibco, #11140), Pen/Strep (6 mL), sodium pyruvate (6 mL), DMSO (9 mL) and FBS (10 mL) (all per 500 mL medium). Medium was changed every 3 days and supernatants were harvested for 2 weeks. Virus was concentrated from supernatants by PEG precipitation and the titer (genome equivalent (GE)/mL) was determined by qPCR. Briefly, supernatants were mixed with 40% PEG solution at a ratio of 4:1, incubated on a shaker at 4° C. overnight and then centrifuged using 50 mL falcon tubes at 4° C. for one hour at 3724 g (RCF). The supernatant was discarded and the centrifugation step was repeated with new supernatant reusing the tubes until all PEG-precipitated supernatant was processed. The pellets were re-suspended in William's E Medium (Gibco, #22551) at a concentration of 10⁷-10⁹ genome equivalents (GE) per mL and frozen at −80° C. DNA copy number calculation was based on a standard curve generated from HBV plasmid dilutions with known concentrations.

HepaRG cells (Biopredic International, Saint-Gregoire, France) were cultured in working growth medium (500 mL Willams E Medium with 50 mL HepaRG Growth supplement from Biopredic, 5 mL Glutamax-I (Gibco, #35050) and 5 mL Pen/Strep) for 2 weeks. After 2 weeks medium was changed to differentiation medium containing 1.8% DMSO (500 mL Willams E Medium with 50 mL HepaRG Growth supplement from Biopredic, 5 mL Pen/Strep, 5 mL Glutamax-I and 9 mL DMSO). Medium was changed twice a week up to 2 weeks. Once fully differentiated, cells were trypsinized and seeded into collagenated 96 well plates (50,000 cells/well in 100 μL) in differentiation medium. Cells were cultured at least 5 days in the 96 well plates before they were infected with HBV.

Infection and Compound Treatment of hepaRG Cells

For HBV infection of differentiated HepaRG cells, differentiation medium was removed and new differentiation medium (120 μL/well) containing 4% PEG-8000 and virus stock (20 to 30 GE/cell) was added. Cells were cultured at 37° C. for 16 to 20 h before medium was removed, cells were washed 4 times with PBS and new differentiation medium (120 μL/well) was added.

At day 4 post infection, medium was removed and 100 μL new differentiation medium was added to each well. 3-fold serial dilutions (5 μL compound to 10 μL DMSO) of Drug A and Drug B were prepared in 100% DMSO starting with 15 μL undiluted compound solution (400-fold concentration of highest test concentration). 5 μL of drug A and B dilutions were then added to 990 μL medium (containing 0.8% DMSO) in a 96 deep well plate in a fashion according to the design shown in Table 3. 100 μL thereof were added to the HepaRG cells with a final DMSO concentration of 1.8%. Drug A tested was Compound 6; Drug B tested was Compound 1, Compound 2, Compound 3, Compound 4 or Compound 5. The concentration ranges tested were 1 nM to 0.01 nM for Compound 6, and 300 nM to 0.412 nM for Compound 1 to 5. Medium was replaced by new medium with compound at day 7 post infection and at day 11 post infection cell supernatants were harvested and directly used for HBV DNA extraction or stored at −20° C. Cell viability of the cells was determined using the cell viability assay described below.

TABLE 3
Plate layout for combinations of Drug A and Drug B

| | 1 | 2 | 3 | 4 | 5 | 6 | 7 | 8 | 9 | 10 | 11 | 12 |
|---|---|---|---|---|---|---|---|---|---|---|---|---|
| a | X | X | X | X | X | X | X | X | X | X | X | X |
| b | X | A1 | A1 + B7 | A1 + B6 | A1 + B5 | A1 + B4 | A1 + B3 | A1 + B2 | A1 + B1 | VC | ETV | CC |
| c | X | A2 | A2 + B7 | A2 + B6 | A2 + B5 | A2 + B4 | A2 + B3 | A2 + B2 | A2 + B1 | VC | ETV | CC |
| d | X | A3 | A3 + B7 | A3 + B6 | A3 + B5 | A3 + B4 | A3 + B3 | A3 + B2 | A3 + B1 | VC | ETV | CC |
| e | X | A4 | A4 + B7 | A4 + B6 | A4 + B5 | A4 + B4 | A4 + B3 | A4 + B2 | A4 + B1 | VC | ETV | CC |
| f | X | A5 | A5 + B7 | A5 + B6 | A5 + B5 | A5 + B4 | A5 + B3 | A5 + B2 | A5 + B1 | VC | ETV | CC |
| g | X | VC | B7 | B6 | B5 | B4 | B3 | B2 | B1 | VC | ETV | CC |
| h | X | X | X | X | X | X | X | X | X | X | X | X |

X: PBS
CC: cell control (uninfected)
VC: virus control
ETV: reference control (200 nM Entecavir)
A1-5: serial dilution of drug A
B1-7: serial dilution of drug B
A1 + B7: example of combination of drug A and B at different ratios

### DNA Extraction

HBV DNA from HepaRG cell supernatants was extracted using the MagNA Pure 96 (Roche) robot. 100 μL of the supernatants were mixed in a processing cartridge with 200 μL MagNA Pure 96 external lysis buffer (Roche, Cat. No. 06374913001) and incubated for 10 minutes. DNA was then extracted using the “MagNA Pure 96 DNA and Viral Nucleic Acid Small Volume Kit” (Roche, Cat. No. 06543588001) and the “Viral NA Plasma SV external lysis 2.0” protocol. DNA elution volume was 50 μL.

qPCR

Quantification of extracted HBV DNA was performed using a Taqman qPCR machine (ViiA7, life technologies). Each DNA sample was tested in duplicate in the PCR. 5 μL of DNA sample were added to 15 μL of PCR mastermix containing 10 μL TaqMan Gene Expression Master Mix (Applied Biosystems, Cat. no. 4369016), 0.5 μL PrimeTime XL qPCR Primer/Probe (IDT, Leuven, Belgium) and 4.5 μL distilled water in a 384 well plate and the PCR was performed using the following settings: UDG Incubation (2 min, 50° C.), Enzyme Activation (10 min, 95° C.) and PCR (40 cycles with 15 sec, 95° for Denaturing and 1 min, 60° C. for annealing and extension). DNA copy numbers were calculated from Cₜ values based on a HBV plasmid DNA standard curve by the ViiA7 software.

### Sequences for TaqMan Primers and Probes (IDT):

Forward core primer (F3_core):
CTG TGC CTT GGG TGG CTT T
Reverse primer (R3_core):
AAG GAA AGA AGT CAG AAG GCA AAA
Taqman probe (P3_core):
56-FAM/AGC TCC AAA/ZEN/TTC TTT ATA AGG GTC
GAT GTC CAT G/3IABkFQ

### Cell Viability Assay

Cell viability of the HBV infected and treated HepaRG cells was determined at day 11 post infection using the CellTiter-Glo® (CTG) Luminescent Cell Viability Assay (Promega, Cat. no. G7572). 100 μL of CTG reagent were added to each well of the cells, incubated for 10 min and 80 μL of each well were transferred to a new white 96 well plate. Luminescence (0.2 sec) was measured using an Envision reader (PerkinElmer).

### Data Analysis

### Isobologram Model

The combination experimental results were analyzed using the model described by Craig et al. (Craig J, Duncan I, Whittaker L and Roberts N. (1990). Antiviral synergy between inhibitors of HIV proteinase and reverse transcriptase. Antiviral. Chem. Chemother. 4:161-166). EC₅₀ values were obtained for compounds used alone and in combination with others. To relate these two values and describe the degree of synergy/additivity/antagonism between them, the Fractional Inhibitory Concentration (FIC) was first calculated and used to generate isobolograms. Briefly, the FIC is the ratio of the EC₅₀ of the drug in combination to the EC₅₀ of the drug on its own:

FIC=ratio[EC_{50 combination}:EC_{50 alone}]

The Combination Index (CI), obtained by adding the FICs of the two compounds, was then used to describe the effect between compounds used in the combinations. A CI<1 means synergism, a CI=1 means additivity and a CI>1 means antagonism. The EC₅₀ calculations of the drug combinations were not accurate if one drug alone already showed high effect levels, thus leading to invalid high CI values. These CI values were considered to be insignificant.

### Results

Combination of Compound 1 with Compound 6, combination of Compound 2 with Compound 6, combination of Compound 3 with Compound 6, combination of Compound 4 with Compound 6, and combination of Compound 5 with Compound 6 were tested for anti-HBV activity in HBV infected differentiated HepaRG cells. The single compound inhibitory activites (EC₅₀) obtained in the combination studies were determined (Table 4).

TABLE 4
Mean EC₅₀ ± SD values for the individual
compounds used in the combination studies

| | | EC₅₀ ± SD (n^{a}) | EC₅₀ ± SD (n^{a}) |
|---|---|---|---|
| Drug A | Drug B | Drug A | Drug B |
| Compound 1 | Compound 6 | 19.9 nM ± 3.9 (3) | 0.110 nM ± 0.004 (3) |
| Compound 2 | Compound 6 | 5.8 nM ± 2.0 (3) | 0.11 nM ± 0.03 (3) |
| Compound 3 | Compound 6 | 7.5 nM ± 3.1 (3) | 0.09 nM ± 0.03 (3) |
| Compound 4 | Compound 6 | 16.2 nM ± 9.4 (3) | 0.07 nM ± 0.02 (3) |
| Compound 5 | Compound 6 | 24.8 nM ± 7.3 (3) | 0.08 nM ± 0.03 (3) |

^{a)}number of independent experiments

The calculation of FIC and CI for above combinations was listed below in Table 5 to 9.

TABLE 5
Mean EC₅₀ values for the combination of Compound
1 and Compound 6 and resulting FIC and CI values

| EC₅₀ (nM) of | EC₅₀ (nM) of | | | |
|---|---|---|---|---|
| combination: | combination: | FIC of | FIC of | |
| compund 1 at fixed | compund 6 at fixed | Com- | Com- | |
| concentration | concentration | pound 6 | pound 1 | CI |
| 0.09 | 0.00 | 1.00 | 0.00 | 1.00 |
| 0.07 | 0.62 | 0.84 | 0.08 | 0.92 |
| 0.07 | 0.62 | 0.76 | 0.08 | 0.84 |
| 0.06 | 1.85 | 0.66 | 0.23 | 0.89 |
| 0.03 | 5.56 | 0.33 | 0.69 | 1.02 |
| 0.00 | 16.67 | 0.00 | 2.07 | 2.07 |
| 0.00 | 8.05 | 0.00 | 1.00 | 1.00 |
| 0.01 | 4.60 | 0.14 | 0.57 | 0.72 |
| 0.04 | 3.45 | 0.43 | 0.43 | 0.86 |
| 0.11 | 0.36 | 1.30 | 0.05 | 1.34 |
| 0.33 | 0.00 | 3.89 | 0.00 | 3.89 |
| 1.00 | 0.00 | 11.66 | 0.00 | 11.66 |

TABLE 6
Mean EC₅₀ values for the combination of Compound
2 and Compound 6 and resulting FIC and CI values

| EC₅₀ (nM) of | EC₅₀ (nM) of | | | |
|---|---|---|---|---|
| combination: | combination: | FIC of | FIC of | |
| compund 2 at fixed | compund 6 at fixed | Com- | Com- | |
| concentration | concentration | pound 6 | pound 2 | CI |
| 0.08 | 0.00 | 1.00 | 0.00 | 1.00 |
| 0.07 | 0.62 | 0.81 | 0.09 | 0.90 |
| 0.06 | 0.62 | 0.70 | 0.09 | 0.79 |
| 0.04 | 1.85 | 0.44 | 0.27 | 0.70 |
| 0.01 | 5.56 | 0.17 | 0.80 | 1.97 |
| 0.00 | 16.67 | 0.00 | 2.40 | 2.40 |
| 0.00 | 6.95 | 0.00 | 1.00 | 1.00 |
| 0.01 | 3.42 | 0.15 | 0.49 | 0.64 |
| 0.04 | 2.39 | 0.46 | 0.34 | 0.80 |
| 0.11 | 0.08 | 1.37 | 0.01 | 1.38 |
| 0.33 | 0.00 | 4.10 | 0.00 | 4.10 |
| 1.00 | n.a. | 12.30 | n.a. | n.a. |

TABLE 7
Mean EC₅₀ values for the combination of Compound
3 and Compound 6 and resulting FIC and CI values

| EC₅₀ (nM) of | EC₅₀ (nM) of | | | |
|---|---|---|---|---|
| combination: | combination: | FIC of | FIC of | |
| compund 3 at fixed | compund 6 at fixed | Com- | Com- | |
| concentration | concentration | pound 6 | pound 3 | CI |
| 0.09 | 0.00 | 1.00 | 0.00 | 1.00 |
| 0.06 | 1.23 | 0.69 | 0.21 | 0.90 |
| 0.04 | 1.23 | 0.42 | 0.21 | 0.63 |
| 0.02 | 3.70 | 0.26 | 0.63 | 0.88 |
| 0.00 | 11.11 | 0.01 | 1.88 | 1.89 |
| 0.02 | 33.33 | 0.18 | 5.65 | 5.83 |
| 0.00 | 5.90 | 0.00 | 1.00 | 1.00 |
| 0.01 | 2.65 | 0.14 | 0.45 | 0.59 |
| 0.04 | 2.67 | 0.43 | 0.45 | 0.88 |
| 0.11 | 0.06 | 1.28 | 0.01 | 1.29 |
| 0.33 | 0.00 | 3.83 | 0.00 | 3.83 |
| 1.00 | n.a. | 11.50 | n.a. | n.a. |

TABLE 8
Mean EC₅₀ values for the combination of Compound
4 and Compound 6 and resulting FIC and CI values

| EC₅₀ (nM) of | EC₅₀ (nM) of | | | |
|---|---|---|---|---|
| combination: | combination: | FIC of | FIC of | |
| compund 4 at fixed | compund 6 at fixed | Com- | Com- | |
| concentration | concentration | pound 6 | pound 4 | CI |
| 0.07 | 0.00 | 1.00 | 0.00 | 1.00 |
| 0.04 | 1.23 | 0.53 | 0.12 | 0.65 |
| 0.03 | 1.23 | 0.40 | 0.12 | 0.52 |
| 0.02 | 3.70 | 0.37 | 0.36 | 0.73 |
| 0.01 | 11.11 | 0.16 | 1.09 | 1.25 |
| 0.00 | 33.33 | 0.00 | 3.26 | 3.26 |
| 0.00 | 10.23 | 0.00 | 1.00 | 1.00 |
| 0.01 | 4.32 | 0.19 | 0.42 | 0.61 |
| 0.04 | 2.72 | 0.56 | 0.27 | 0.83 |
| 0.11 | 0.02 | 1.68 | 0.00 | 1.69 |
| 0.33 | 0.00 | 5.05 | 0.00 | 5.05 |
| 1.00 | n.a. | 15.16 | n.a. | n.a. |

TABLE 9
Mean EC₅₀ values for the combination of Compound
5 and Compound 6 and resulting FIC and CI values

| EC₅₀ (nM) of | EC₅₀ (nM) of | | | |
|---|---|---|---|---|
| combination: | combination: | FIC of | FIC of | |
| compund 5 at fixed | compund 6 at fixed | Com- | Com- | |
| concentration | concentration | pound 6 | pound 5 | CI |
| 0.08 | 0.00 | 1.00 | 0.00 | 1.00 |
| 0.05 | 1.23 | 0.68 | 0.08 | 0.75 |
| 0.04 | 1.23 | 0.48 | 0.08 | 0.56 |
| 0.04 | 3.70 | 0.47 | 0.23 | 0.70 |
| 0.02 | 11.11 | 0.22 | 0.69 | 0.91 |
| 0.00 | 33.33 | 0.00 | 2.06 | 2.06 |
| 0.00 | 16.19 | 0.00 | 1.00 | 1.00 |
| 0.01 | 7.92 | 0.15 | 0.49 | 0.64 |
| 0.04 | 3.76 | 0.46 | 0.23 | 0.69 |
| 0.11 | 0.08 | 1.38 | 0.01 | 1.38 |
| 0.33 | 0.00 | 4.14 | 0.00 | 4.14 |
| 1.00 | n.a. | 12.41 | n.a. | n.a. |

The interaction between Compound 1 and Compound 6 was analyzed using the Isobologram model (FIG. 1). The FIC values for the combination of Compound 1 and Compound 6 were plotted one against the other for each of the experiments. The analysis showed that most of the CI values were <1. Therefore, the combination of Compound 1 and Compound 6 was synergistic.

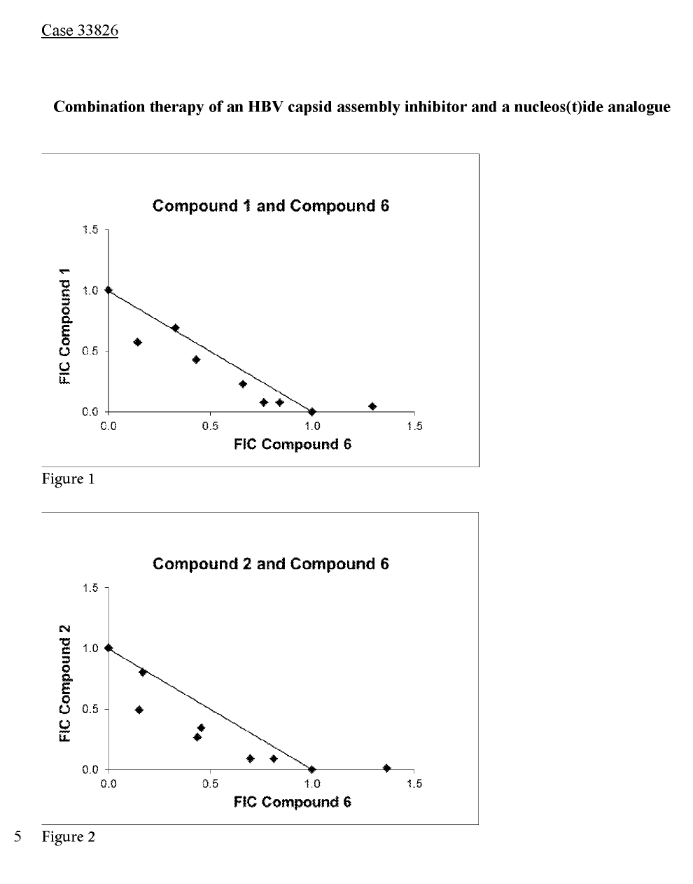

The interaction between Compound 2 and Compound 6 was analyzed using the Isobologram model (FIG. 2). The FIC values for the combination of Compound 2 and Compound 6 were plotted one against the other for each of the experiments. The analysis showed that most of the CI values were <1. Therefore, the combination of Compound 2 and Compound 6 was synergistic.

The interaction between Compound 3 and Compound 6 was analyzed using the Isobologram model (FIG. 3). The FIC values for the combination of Compound 3 and Compound 6 were plotted one against the other for each of the experiments. The analysis showed that most of the CI values were <1. Therefore, the combination of Compound 3 and Compound 6 was synergistic.

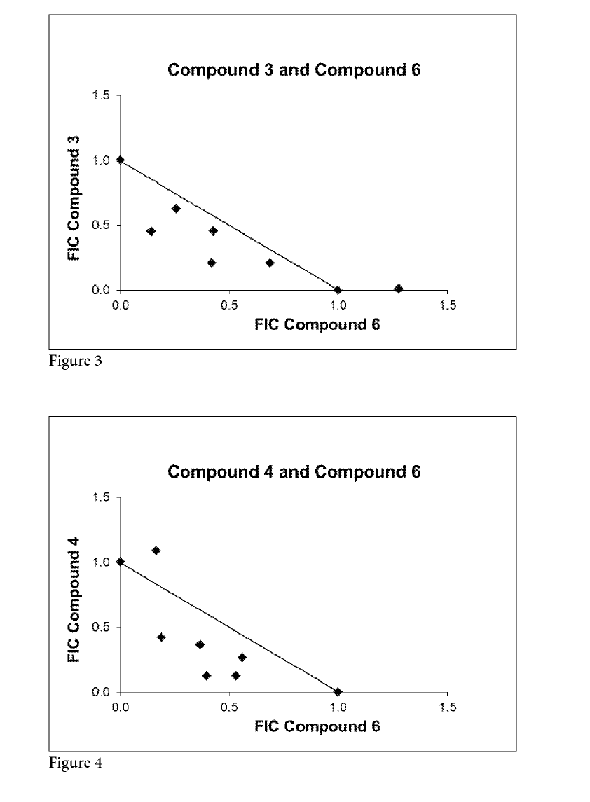

The interaction between Compound 4 and Compound 6 was analyzed using the Isobologram model (FIG. 4). The FIC values for the combination of Compound 4 and Compound 6 were plotted one against the other for each of the experiments. The analysis showed that most of the CI values were <1. Therefore, the combination of Compound 4 and Compound 6 was synergistic.

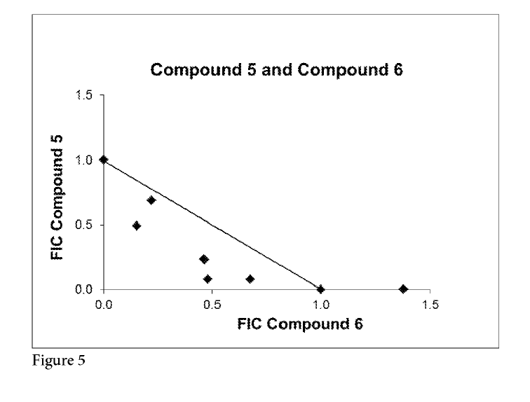

The interaction between Compound 5 and Compound 6 was analyzed using the Isobologram model (FIG. 5). The FIC values for the combination of Compound 5 and Compound 6 were plotted one against the other for each of the experiments. The analysis showed that most of the CI values were <1. Therefore, the combination of Compound 5 and Compound 6 was synergistic.

None of the above combinations had a significant effect on cell viability.

1. A pharmaceutical composition comprising an HBV capsid assembly inhibitor and a nucleoside or nucleotide analogue, in a pharmaceutically acceptable carrier.

2. The pharmaceutical composition according to claim 1, wherein the HBV capsid assembly inhibitor is a compound of formula (I) 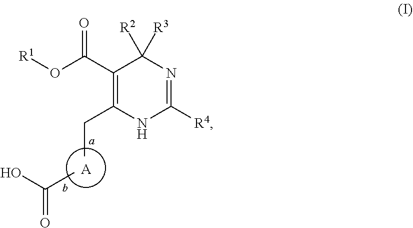
wherein:
R¹ is C₁₋₆alkyl or trifluoromethyl-CₓH₂ₓ—, wherein x is 1, 2, 3, 4, 5 or 6;
one of R² and R³ is phenyl, which is once or twice or three times substituted by C₁₋₆alkyl, cyano or halogen; and the other one is hydrogen or deuterium;
R⁴ is phenyl, thiazolyl, oxazolyl, imidazolyl, thienyl or pyridinyl, which is unsubstituted or substituted by C₁₋₆alkyl, C₁₋₆alkylsulfanyl, halogen or cycloalkyl, wherein C₁₋₆alkyl can be further optionally substituted with halogen;
A is 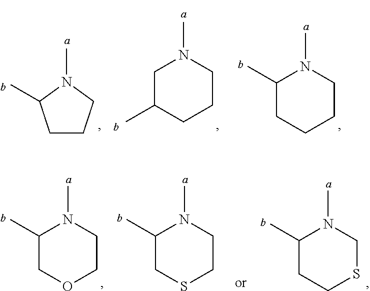
which is unsubstituted or substituted by groups selected from C₁₋₆alkyl, deuterium and halogen;
or pharmaceutically acceptable salt, or enantiomer, or diastereomer thereof.

3. The pharmaceutical composition according to claim 2, wherein the HBV capsid assembly inhibitor is (S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carb oxylic acid.

4. The pharmaceutical composition according to claim 1, wherein the HBV capsid assembly inhibitor is a compound of formula (II): 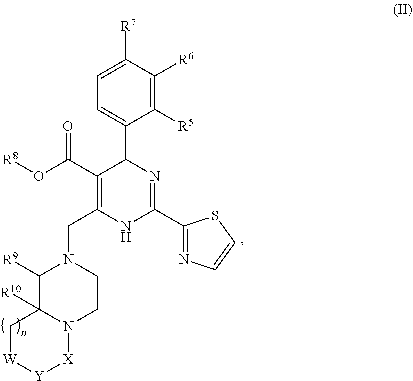
wherein:
R⁵ is hydrogen, halogen or C₁₋₆alkyl;
R⁶ is hydrogen or halogen;
R⁷ is hydrogen or halogen;
R⁸ is C₁₋₆alkyl;
R⁹ is hydrogen, hydroxyC₁₋₆alkyl, aminocarbonyl, C₁₋₆alkoxycarbonyl or carboxy;
R¹⁰ is hydrogen, C₁₋₆alkoxycarbonyl or carboxy-CₘH₂ₘ—;
X is carbonyl or sulfonyl;
Y is —CH₂—, —O— or —N(R¹¹)—,
wherein R¹¹ is hydrogen, C₁₋₆alkyl, haloC₁₋₆alkyl, C₃₋₇cycloalkyl-CₘH₂ₘ—, C₁₋₆alkoxycarbonyl-CₘH₂ₘ—, —CₜH₂ₜ—COOH, -haloC₁₋₆alkyl-COOH, —(C₁₋₆alkoxy)C₁₋₆alkyl-COOH, —C₁₋₆alkyl-O—C₁₋₆alkyl-COOH, —C₃₋₇cycloalkyl-CₘH₂ₘ—COOH, —CₘH₂ₘ—C₃₋₇cycloalkyl-COOH, hydroxy-CₜH₂ₜ—, carboxyspiro[3.3]heptyl or carboxyphenyl-CₘH₂ₘ—, carboxypyridinyl-CₘH₂ₘ—;
W is —CH₂—, —C(C₁₋₆alkyl)₂-, —O— or carbonyl;
n is 0 or 1;
m is 0-7;
t is 1-7;
or pharmaceutically acceptable salt, or enantiomer or diastereomer thereof.

5. The pharmaceutical composition according to claim 1, wherein the HBV capsid assembly inhibitor is:
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid; or
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid;
or a pharmaceutically acceptable salt, enantiomer or diastereomer thereof.

6. The pharmaceutical composition according to claim 1, wherein the nucleoside or nucleotide analogue is Entecavir, Lamivudine, Adefovir dipivoxil, Telbivudine, Clevudine, Tenofovir disoproxil or Tenofovir disoproxil fumarate.

7. The pharmaceutical composition according to claim 6, wherein the nucleoside or nucleotide analogue is Entecavir.

8. The pharmaceutical composition according to claim 1, wherein the composition consists of an HBV capsid assembly inhibitor and a nucleoside or nucleotide analogue, in a pharmaceutically acceptable carrier.

9. A pharmaceutical composition comprising a compound selected from the group consisting of:
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Entecavir;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Entecavir;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Entecavir;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Entecavir;
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Entecavir;
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Lamivudine;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Lamivudine;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Lamivudine;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Lamivudine;
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Lamivudine;
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Adefovir dipivoxil;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Adefovir dipivoxil;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Adefovir dipivoxil;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Adefovir dipivoxil;
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Adefovir dipivoxil;
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Telbivudine;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Telbivudine;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Telbivudine;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Telbivudine;
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Telbivudine;
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Clevudine;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Clevudine;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Clevudine;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Clevudine;
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Clevudine;
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Tenofovir disoproxil;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Tenofovir disoproxil;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Tenofovir disoproxil;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Tenofovir disoproxil;
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Tenofovir disoproxil;
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Tenofovir disoproxil fumarate;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Tenofovir disoproxil fumarate;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Tenofovir disoproxil fumarate;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Tenofovir disoproxil fumarate; or
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Tenofovir disoproxil fumarate;
in a pharmaceutically acceptable carrier.

10. A pharmaceutical composition according to claim 9 consists of:
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Entecavir;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Entecavir;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid and Entecavir;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Entecavir; or
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Entecavir;
in a pharmaceutically acceptable carrier.

11-19. (canceled)

20. A kit comprising a container comprising an HBV capsid assembly inhibitor and a nucleoside or nucleotide analogue.

21. The kit according to claim 20, further comprising a sterile diluent.

22. The kit according to claim 20, further comprising a package insert comprising printed instructions directing the use of a combined treatment of an HBV capsid assembly inhibitor and a nucleoside or nucleotide analogue as a method for treatment or prophylaxis of hepatitis B virus infection.

23. The kit according to claim 20, wherein the HBV capsid assembly inhibitor is:
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-2,2-dimethyl-propanoic acid;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid; or
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid;
or a pharmaceutically acceptable salt, enantiomer or diastereomer thereof.

24. The kit according to claim 20, wherein the nucleoside or nucleotide analogue is Entecavir, Lamivudine, Adefovir dipivoxil, Telbivudine, Clevudine, Tenofovir disoproxil or Tenofovir disoproxil fumarate. Particularly the nucleoside analogue is Entecavir.

25. The kit according to claim 20, wherein the HBV capsid assembly inhibitor and the nucleoside or nucleotide analogue used in the container are
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Entecavir;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Entecavir;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Entecavir;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Entecavir; or
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Entecavir.

26. A method for the treatment or prophylaxis of hepatitis B virus infection, comprising administration to a subject with an effective first amount of an HBV capsid assembly inhibitor, or a pharmaceutically acceptable salt, enantiomer or diastereomer thereof; and a second amount of a nucleoside or nucleotide analogue; or vice versa.

27. The method according to claim 26, wherein the HBV capsid assembly inhibitor is:
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid; or
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid;
or a pharmaceutically acceptable salt, enantiomer or diastereomer thereof.

28. The method according to claim 26, wherein the nucleoside or nucleotide analogue is Entecavir, Lamivudine, Adefovir dipivoxil, Telbivudine, Clevudine, Tenofovir disoproxil or Tenofovir disoproxil fumarate.

29. The method according to claim 26, wherein the HBV capsid assembly inhibitor and the nucleoside or nucleotide analogue used are selected from:
(S)-4-[(R)-6-(2-Chloro-4-fluoro-phenyl)-5-methoxycarbonyl-2-thiazol-2-yl-3,6-dihydro-pyrimidin-4-ylmethyl]-morpholine-3-carboxylic acid and Entecavir;
3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Entecavir;
3-[(8aS)-7-[[(4R)-4-(2-chloro-3-fluoro-phenyl)-5-ethoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Entecavir;
4-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6, 8, 8a-tetrahydro-1H-imidazo[1,5-a]pyrazin-2-yl]-3,3-dimethyl-butanoic acid and Entecavir; or
3-[(8aS)-7-[[(4R)-4-(2-chloro-4-fluoro-phenyl)-5-methoxycarbonyl-2-thiazol-2-yl-1,4-dihydropyrimidin-6-yl]methyl]-3-oxo-5,6,8, 8a-tetrahydro-1H-imidazo[1,5-a]pyrazin-2-yl]-2,2-dimethyl-propanoic acid and Entecavir.

30-31. (canceled)


SEQUENCE LISTING (3 sequences)

SEQ ID NO: 1 - DNA, 19 bp, Hepatitis B virus
ctgtgccttgggtggcttt

SEQ ID NO: 2 - DNA, 24 bp, Hepatitis B virus
aaggaaagaagtcagaaggcaaaa

SEQ ID NO: 3 - DNA, 34 bp, Hepatitis B virus
agctccaaattctttataagggtcgatgtccatg